(19) United States
(12) Patent Application Publication
(10) Pub. No.: US 2002/0166559 A1
(43) Pub. Date: Nov. 14, 2002

(54) Central venous catheter protector

(76) Inventor: Paula Fleming, Pearland, TX (US)

Correspondence Address: CONLEY ROSE & TAYON, P.C., P. O. BOX 3267, HOUSTON, TX, 77253-3267, US

(21) Appl. No.: 10/105,947

(22) Filed: Mar. 25, 2002

Related U.S. Application Data
(60) Provisional application No. 60/278,791, filed on Mar. 26, 2001.

(51) Int. Cl. A61B 19/08

(52) U.S. Cl. 128/853

(57) ABSTRACT

A protector for protecting a central venous catheter disposed on a patient. The protector includes a plastic sheet sized to cover the central venous catheter and area on the patient around the central venous catheter. A pad having an absorbent layer of material and a cloth layer of material is attached to the plastic sheet. A pocket is preferably formed in the absorbent layer for receiving a portion of the central venous catheter.

### CROSS-REFERENCE TO RELATED APPLICATIONS

This application claims the benefit under 35 U.S.C. §119(e) of U.S. Provisional Patent Application No. 60/278,791, filed Mar. 26, 2001, hereby incorporated herein by reference.

### STATEMENT REGARDING FEDERALLY SPONSORED RESEARCH OR DEVELOPMENT

Not applicable.

### BACKGROUND OF THE INVENTION

A central venous catheter (CVC) is used to give a patient intravenous fluids and is worn for long periods of time by patients requiring dialysis, chemotherapy, or other injections into the bloodstream. The CVC includes a catheter implanted into the patient, often in the shoulder or arm, and includes a pair of lumens. One lumen is used for blood sampling and the other lumen is for injecting fluids into the bloodstream. Each lumen includes a tube, typically four to six inches long, extending from the lumen to the catheter.

The catheter, lumens, and the site on the body around the catheter and lumens must be kept dry. Therefore patients who have one or more implanted CVCs are required to keep the CVC and body area around the CVC dry to prevent infection. Each patient who has such a catheter must attend a class for the care of the CVC. The CVC has a dressing that typically is changed weekly. Thus, a covering is taped over the CVC so that the dressing can be changed. Generally, the lumens must be flushed and cleaned every day. A needle type device is inserted into the lumen and a fluid is injected to clean the passageways, particularly of any a blood, to keep the lumens from becoming clogged.

In the past, Saran Wrap has been used to cover the CVC and the area around the CVC. The Saran Wrap is cut into a square and, with the lumens tucked underneath the Saran Wrap, the Saran Wrap is taped to the skin of the body around the CVC. The Saran Wrap is not strong and often punctures causing the CVC and body area around the CVC to get wet, as for example during bathing. Further, the CVC under the Saran Wrap is very uncomfortable to the wearer.

One disadvantage is that the Saran Wrap is not fitted. The Saran Wrap often is not cut to shape for covering the catheter and lumens. The Saran Wrap never seems to cover the whole site.

Another problem with the prior art is that the lumens fall out of the Saran Wrap. The lumens often become loose under the Saran Wrap and thus do not feel secure to the patient. There is also concern that while taking a bath, the lumens might come out of the Saran Wrap. This gives the patent an insecure feeling while bathing. Thus, some patients are concerned about taking a shower or bath and only take a sponge bath. Also, there is a fear of a hole being punctured in the Saran Wrap permitting leakage.

The Saran Wrap also provides no protection because it is too thin and has no rigidity. The Saran Wrap does not absorb force if the catheter area is bumped.

Another conventional application is a paper, called Aqua Guard, which is disposable and used as a covering. It, however, is very costly.

The present invention overcomes the deficiencies of the prior art.

### SUMMARY OF THE INVENTION

A protector for protecting a central venous catheter disposed on a patient. The protector includes a plastic sheet sized to cover the central venous catheter and area on the patient around the central venous catheter. A pad having an absorbent layer of material and a cloth layer of material is attached to the plastic sheet. A pocket is preferably formed in the absorbent layer for receiving a portion of the central venous catheter.

The protector has many advantages over the prior art. The protector may be washed in a washing machine and be reused. The protector keeps the catheter and lumens dry. The padding also provides absorbency such that even if water were to leak around the tape, a minimum amount of water may be absorbed by the pad. Further the padding provides protection to the catheter and lumens if they are touched or bumped. The protector is easily applied over the CVC and is very comfortable to the wearer.

Other objects and advantages of the invention will appear from the following description.

### BRIEF DESCRIPTION OF THE DRAWINGS

For a detailed description of a preferred embodiment of the invention, reference will now be made to the accompanying drawings wherein:

FIG. 1 is a side elevation view of the plastic sheet side of the protector;

FIG. 2 is a side elevation view of the padded side of the protector; and

FIG. 3 is a cross-sectional view taken at plane 3-3 in FIG. 2.

### DESCRIPTION OF PREFERRED EMBODIMENTS

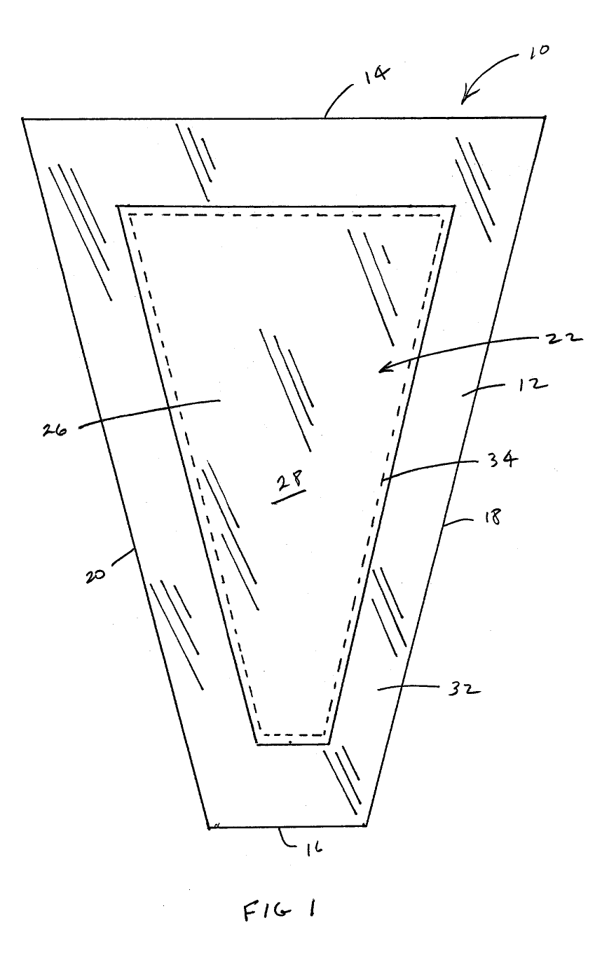

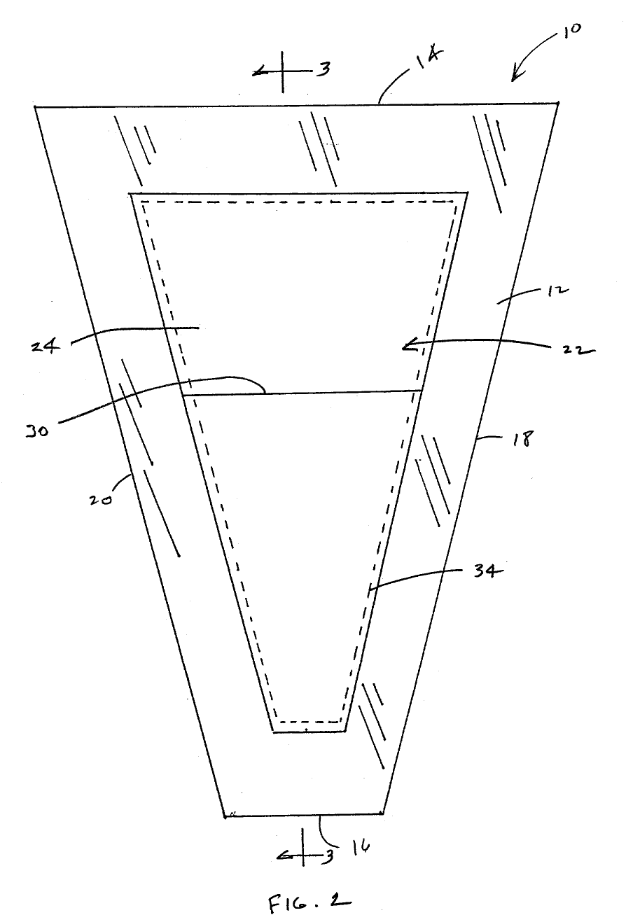

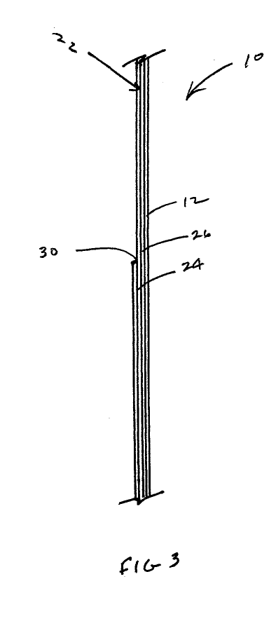

Referring to FIGS. 1-3, there is shown a preferred embodiment of the CVC protector 10. Protector 10 includes a plastic sheet 12 cut in appropriate shape to cover the catheter and lumens of the CVC and that portion of the body around the CVC. The shape of protector 10 is dependent upon the body area to be covered, such as on the shoulder or arm. Therefore the protector 10 may be of any shape and, for the shoulder, is preferably an elongated trapezoid where the longer parallel side 14 is adjacent the top of the shoulder of the patient and the shorter parallel side 16 is on a lower portion of the shoulder. The angled sides 18, 20 extend between the ends of parallel sides 14, 16. The plastic sheet 12 must be waterproof and is preferably made of a vinyl plastic having a thickness which causes the plastic to have some degree of rigidity, a greater rigidity that Saran Wrap.

A pad 22 is attached, such as by sewing, to the plastic sheet 12. Standard thread 34 may be used to sew the pad 22 to the plastic sheet 12. The pad 22 includes a backing which is preferably made of an absorbent cloth and still more preferably made of an absorbent cloth such as that used for towels. A terrycloth 24 has been found to be particularly preferred because of its absorbency and softness. Further, the pad 22 may include one or more additional cloth layers 26 to add additional padding. The additional cloth layers 26 are attached to the terrycloth 24, such as by sewing, with the terrycloth layer 24 preferably being adapted to be adjacent the patient's skin. The additional cloth layer 26 may be decorative on that side 28 adjacent the plastic sheet 12 so that it can be seen by the wearer. The pad 22 also serves the purpose of covering the CVC so that the CVC cannot be seen by the patient.

The pad 22 further includes a pocket 30 for receiving and holding the lumens. The pocket 30 is also made of an absorbent cloth and preferably made of the same terrycloth material 24. The pocket is formed by sewing the absorbent cloth onto the base terrycloth material 24 of the pad 22. The pocket 30 has dimensions so as to receive the lumens.

In production, the additional padding material 26 is first sewn onto the base absorbent material 24 and the pocket material 30 is sewn onto the terrycloth material 24 to form pad 22. The pad 22 is then sewn onto the plastic sheet 12. It should be appreciated that the pad 22 and plastic sheet 12 may be sewn all together at the same time.

During use, as for example on a CVC disposed in the shoulder of a patient, the lumens are inserted into the pocket 20 of the pad 22 including substantially, if not all, of the tubes extending from the lumen to the catheter. The upper portion of the pad 22 is then placed over the catheter to cover the catheter. The protector 10 serves as patch over the CVC. The extending border 32 of the plastic sheet 12 extending beyond the pad 22 is then taped to the skin of the patient preferably using a waterproof tape. Any waterproof tape may be used to tape protector 10 onto the patient. One preferred tape is the Nexcare waterproof hypo-allergenic tape. The tape is typically placed around the border 32 and skin around the collarbone area of the patient.

The protector 10 has many advantages over the prior art. Protector 10 may be washed in a washing machine and be reused over and over again such as for several months.

The protector 10 keeps the catheter and lumens dry. The padding 22 also provides absorbency. Thus, even if water were to leak around the tape, a minimum amount of water may be absorbed by the pad 22 prior to reaching the catheter or lumens.

Further the padding 22 provides protection to the catheter and lumens if they are touched or bumped. The protector 10 is easily applied over the CVC and is very comfortable to the wearer.

While a preferred embodiment of the invention has been shown and described, modifications thereof can be made by one skilled in the art without departing from the spirit of the invention.

What is claimed is:

1. An apparatus to cover a catheter, comprising:
a plastic sheet; and
a pad sized to cover the catheter and attached to the sheet.

2. The apparatus of claim 1 wherein the pad includes an absorbent material.

3. The apparatus of claim 1 further including a pocket in the pad.

4. The apparatus of claim 1 wherein the pad includes multiple layers of cloth.

5. The apparatus of claim 1 wherein the plastic sheet forms a border around the pad for taping the plastic sheet.

6. A protector for protecting a central venous catheter disposed on a patient, comprising:
a plastic sheet sized to cover the central venous catheter and area on the patient around the central venous catheter;
a pad having an absorbent layer of material and a cloth layer of material; and
a pocket formed in the absorbent layer for receiving a portion of the central venous catheter.

7. The apparatus of claim 1 wherein the pad includes a backing material.

8. An apparatus to cover a catheter, comprising:
a substantially waterproof and puncture-resistant sheet; and
a pad attached to said sheet and sized to cover the catheter.

9. The apparatus of claim 8 wherein the pad includes an absorbent material.

10. The apparatus of claim 8 further including a pocket in the pad.

11. The apparatus of claim 8 wherein the pad includes multiple layers of terrycloth.

12 The apparatus of claim 8 wherein the pad includes a backing material.

13. The apparatus of claim 8 wherein the substantially waterproof and puncture-resistant sheet forms a border around the pad for taping said sheet.

14. A protector for protecting a central venous catheter disposed on a patient, the catheter having lumens, the protector comprising:
a substantially waterproof and puncture-resistant sheet sized to cover the central venous catheter and area on the patient around the central venous catheter;
a pad having an absorbent layer of material and a backing material; and
a pocket formed in the absorbent layer for receiving the lumens from the central venous catheter.

15. The protector of claim 14 wherein said pad is washable.

16. The protector of claim 14 further including waterproof, hypo-allergenic tape attached to said sheet.

17. The protector of claim 14 wherein said pad is inside said sheet next to the patient.

18. The protector of claim 14 wherein said sheet is made of vinyl plastic.
